(19) United States
(12) Patent Application Publication
(10) Pub. No.: US 2023/0240886 A1
(43) Pub. Date: Aug. 3, 2023

(54) Fillable Palm Cooling Device

(71) Applicant: Kyle Michael Sela, Sun Valley, CA (US)

(72) Inventor: Kyle Michael Sela, Sun Valley, CA (US)

(21) Appl. No.: 18/160,539

(22) Filed: Jan. 27, 2023

Related U.S. Application Data
(60) Provisional application No. 63/305,152, filed on Jan. 31, 2022.

(51) Int. Cl. A61F 7/10

(52) U.S. Cl. CPC A61F 7/10; A61F 2007/0036

(57) ABSTRACT

A fillable palm cooling device is described. The fillable palm cooling device is configured to be filled with a cool substance, and includes at least a first endcap configured to be removed to enable the fillable tube to be filled with the cool substance. The fillable tube is configured to be held in palms of both hands of a person to cool the palms of both hands of the person when the fillable tube is filled with the cool substance.

### PRIORITY

This application claims priority to U.S. Provisional Pat. Application Serial No. 63/305,152, filed on Jan. 31, 2022, the disclosure of which is incorporated by reference herein.

### BACKGROUND

As a person’s body temperature increases from exercise or environmental exposure, fatigue increases. Heat causes fatigue in two main ways: 1) increased temperature in working muscle leads to a decreased ability to contract, and 2) this increase in temperature also leads to a decrease in motivation to continue working. Humans, much like many mammals, have portions of their body covered by glabrous skin. Glabrous skin is hairless portions of skin found on the palms of the hands, soles of the feet and hairless portions of the face. Underneath glabrous skin are special networks of blood vessels called arteriovenous anastomoses (AVAs). AVAs are unique in that they are direct connections of arteries to veins with no capillary beds in between. This direct connection indicates that AVAs have a primary role in temperature regulation because the surface temperature of the glabrous skin cools or heats the blood coursing through the AVAs below. With no capillaries involved that blood is immediately returned to the system and decreases or increases core temperature.

### BRIEF DESCRIPTION OF THE DRAWINGS

The detailed description is described with reference to the accompanying figures.

FIG. 1 depicts an example of a fillable palm cooling device in accordance with one or more implementations.

FIG. 2 depicts an additional example of a fillable palm cooling device in accordance with one or more implementations.

FIG. 3 depicts an additional example of a fillable palm cooling device in accordance with one or more implementations.

FIG. 4 depicts an additional example of a fillable palm cooling device in accordance with one or more implementations.

FIG. 5 depicts an additional example of a fillable palm cooling device in accordance with one or more implementations.

FIG. 6 depicts an additional example of a fillable palm cooling device in accordance with one or more implementations.

FIG. 7 depicts an additional example of a fillable palm cooling device in accordance with one or more implementations.

FIG. 8A depicts an additional example of a fillable palm cooling device in accordance with one or more implementations

FIG. 8B depicts an additional example of a fillable palm cooling device in accordance with one or more implementations

### DETAILED DESCRIPTION

### Overview

To solve these problems, a fillable palm cooling device is described. The fillable palm cooling device is configured to be held in the palms of both hands of a person in order to cool both of the person’s hands with one device. Cooling as much glabrous skin surface area at one time is an important factor when attempting to regulate body temperature. Research has shown that purposely cooling the palms as body temperature increases will mitigate the fatigue factors described above. This delay in fatigue has been shown to increase work volume both in single session situations and over the course of a training program period lasting weeks. Research has shown that the most effective temperature range for palm cooling is between 45 to 60° F.

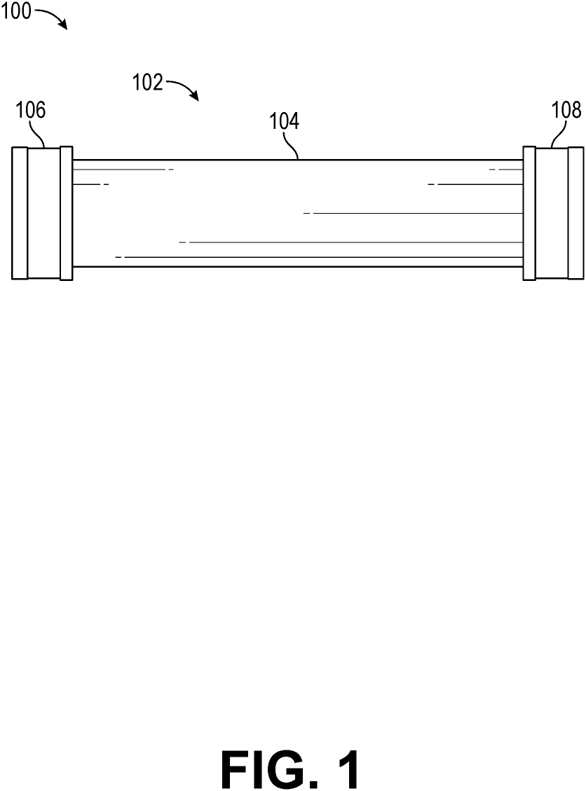

FIG. 1 depicts an example 100 of a fillable palm cooling device 102 in accordance with one or more implementations. In this example, the fillable palm cooling device 102 includes a fillable tube 104, a first endcap 106, and a second endcap 108. In one or more examples, one or both of the first endcap 106 and the second endcap 108 are removable so that the fillable tube 104 can be filled with a cool substance, such as cool water. The first endcap 106 and the second endcap 108 create a watertight seal within the fillable tube 104. In one or more implementations, the endcaps 106 and 108 are made of a flexible rubber. However, the endcaps may also be configured as threaded lids, silicone seals, cork, and so forth. Alternatively, in one or more implementations the fillable palm cooling device 102 includes a single removeable endcap that can be removed so that the fillable tube can be filled with a cool liquid. Notably, the fillable palm cooling device can also be configured in other ways to enable the fillable tube 104 to be filled with a cool liquid.

Generally, the fillable tube 104 has a length which enables the fillable tube 104 to be held in the palms of both hands of the user at the same time. By way of example and not limitation, the fillable tube 104 can be 12 inches long with a diameter of 2.375 inches. The fillable tube 104 is hollow which enables the fillable tube 104 to be filled with a cool substance, such as cool water. In a non-limiting example, the outer circumference of the cooling portion of the tube is 7.5 inches, and the wall of the tube is 0.154 inches thick. It is to be appreciated, however, that the dimensions of the fillable tube can vary without departing from the spirit or scope of the described techniques.

Generally, the fillable tube 104 is made of a material which enables the tube to be cold when filled with a cool liquid, and also to resist the rise in temperature that the palms of the person’s hands impart on the fillable tube 104. In one or more implementations, the fillable tube 104 is made of aluminum. It is to be appreciated, however, that other materials can be utilized instead of aluminum without departing from the spirit or the scope of the described techniques. It is beneficial if the cooling device can maintain its temperature for as long as possible each use before having to be re-cooled. Materials and substances have a physical property called specific heat capacity which is a measure of how much energy it takes to raise the temperature of the material by unit mass. Notably, aluminum has a high specific heat capacity (0.902 J/g C) compared to other metals. Moreover, when the aluminum tube is filled with cool water, the water has an even higher specific heat capacity (4.179 J/g C). Thus, when the fillable tube 104 is made from aluminum and filled with cool water, the aluminum tube and the cool water work in tandem to resist the rise in temperature that the palms of the hands are imparting on the tube.

In example 100, the fillable palm cooling device 102 is implemented with a straight fillable tube 104. In other implementations, however, the fillable tube 104 of the fillable palm cooling device 102 can be configured in a different shape which still enables the fillable tube 104 to be held in the palms of both hands of a person, e.g., a curved fillable tube, an angled fillable tube, or a circular fillable tube.

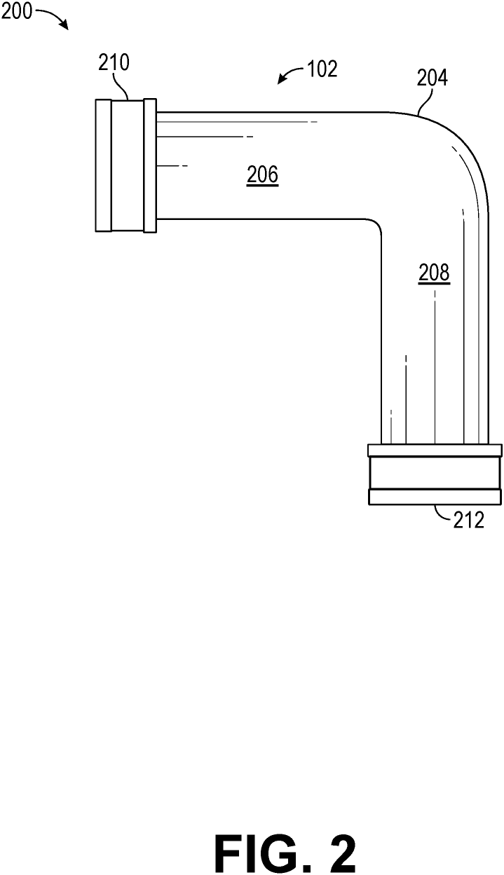

Consider, for example, FIG. 2 which depicts an additional example 200 of a fillable palm cooling device 102. In example 200, the fillable palm cooling device 102 includes a fillable tube 204 which is angled or curved, and includes a first portion 206 that is configured to be held in a first palm of a first hand of a person and a second portion 208 that is configured to be held in a second palm of a second hand of the person. The fillable palm cooling device 102 also includes a first endcap 210 and a second endcap 212. In this example, both the first endcap 210 and the second endcap 212 are removable so that the fillable tube 204 can be filled with a cool substance, such as cool water. The first endcap 210 and the second endcap 212 create a watertight seal within the fillable tube 204. In one or more implementations, the endcaps 210 and 212 are made of a flexible rubber. However, the endcaps may also be configured as threaded lids, silicone seals, cork, and so forth. Alternatively, in one or more implementations the fillable palm cooling device 102 includes a single removeable endcap that can be removed so that the fillable tube can be filled with a cool liquid.

The fillable palm cooling device can be cooled in at least two different ways, by either externally cooling the device or internally cooling the device. External cooling comes from the environment that the fillable palm cooling device is placed in. For example, the fillable palm cooling device can be cooled to a suitable cooling temperature by placing the device in a refrigerator, a cool room, submersion in cold water, and so forth. However, in many cases, a suitable environment for cooling the device may not be readily available. For instance, if a person is using the fillable palm cooling device at a commercial fitness facility, and the temperature of the device eventually exceeded the cooling temperature range (e.g., greater than 60° F.), there may be no practical way to quickly re-cool the device.

However, to solve this problem, the fillable palm cooling device described herein can also be cooled internally by refilling the device with a cool substance, such as cool water. Notably, cool water is often readily available in water fountains and sinks. The cool water has the capacity to cool the aluminum fillable tube of the fillable palm cooling device in seconds without warming the water much (due to differences in specific heat capacity). There are many different ways in which the fillable cooling device can be designed for re-filling and re-cooling, including by way of example and not limitation, with removable endcaps on each end of the fillable tube, a removable endcap on just one end of the fillable tube, a threaded lid on one or both ends of the fillable tube, a removable plug positioned on the body of the fillable tube, and so forth. In this way, the fillable cooling device can be emptied and refilled with new cool water.

In one or more implementations, the fillable palm cooling device further comprises an indicator mechanism to notify the user if the fillable palm cooling device is within a cooling temperature range that is suitable to cool the user’s palms. By way of example and not limitation, the cooling temperature range may be between 45 and 60° F. Notably, other cooling temperature ranges may be utilized, which vary slightly higher or lower from this cooling temperature range, without departing from the spirit or scope of the described devices and techniques. Alternatively or additionally, the indicator mechanism may alert the user if the fillable palm cooling device exceeds the cooling temperature range (e.g., too warm) in order to alert the user that the fillable tube should be re-filled with a cool substance or placed in a cool environment. The indicator mechanism, for example, may be a visual indicator (e.g., a light source) which notifies the user when the fillable palm cooling device is within the palm cooling temperature range. For example, the fillable palm cooling device may include a light source that turns on when the fillable palm cooling device is within the cooling temperature range for palm cooling. Alternatively, the light source can turn on when the temperature of the fillable palm cooling device exceeds the cooling temperature range for palm cooling, e.g.., the temperature exceeds 60° F. In some cases, the light source may be configured to change colors to indicate different states. For example, the light source may turn green when the fillable palm cooling device is within the cooling temperature range, and turn red when the fillable palm cooling device is outside of the cooling temperature range, e.g., too hot. The indicator mechanism may also be configured in other ways without departing from the spirit or scope of the described techniques, such as an audible output that beeps that when the fillable palm cooling device is outside of the cooling temperature range, a message that appears on the fillable palm cooling device, or a thermometer display that indicates the current temperature of the fillable palm cooling device.

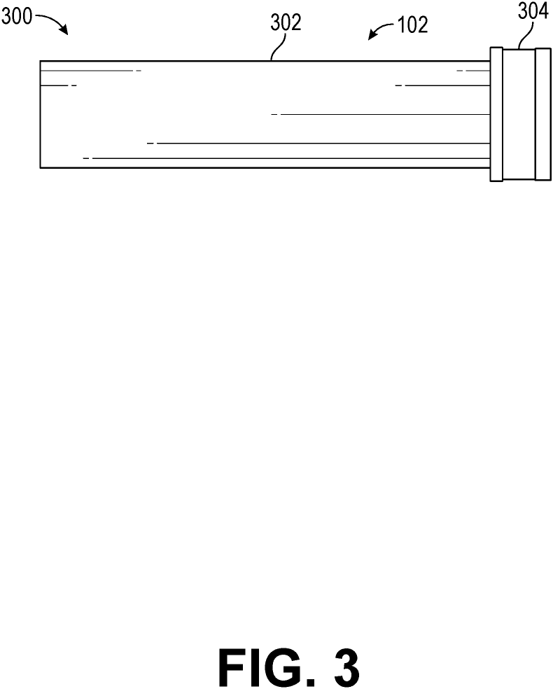

FIG. 3 depicts an additional example 300 of a fillable palm cooling device in accordance with one or more implementations. In this example, the fillable palm cooling device 102 includes a fillable tube 302 and a single endcap 304 which is removable so that the fillable tube 302 can be filled with a cool substance, such as cool water.

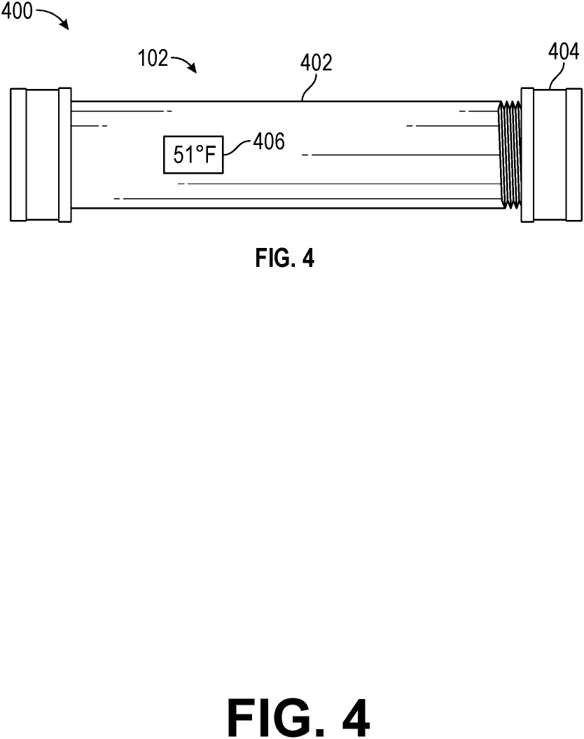

FIG. 4 depicts an additional example 400 of a fillable palm cooling device in accordance with one or more implementations. In this example, the fillable palm cooling device 102 includes a fillable tube 402 and a single threaded lid 404 which can be screwed on and off of the fillable tube 402. For example, the threaded lid 404 can be removed from the fillable tube 402 by unscrewing the threaded lid 404 from the fillable tube 402 so that the fillable tube 402 can be filled with a cool substance, such as cool water. In this example, the fillable palm cooling device 102 is further configured with an indicator mechanism 406. The indicator mechanism 406, in this example, provides a notification of a current temperature of the fillable palm cooling device 102. In this example, the indicator mechanism 406 indicates that the current temperature of the fillable palm cooling device is 51° F., which in some examples is within the cooling temperature range.

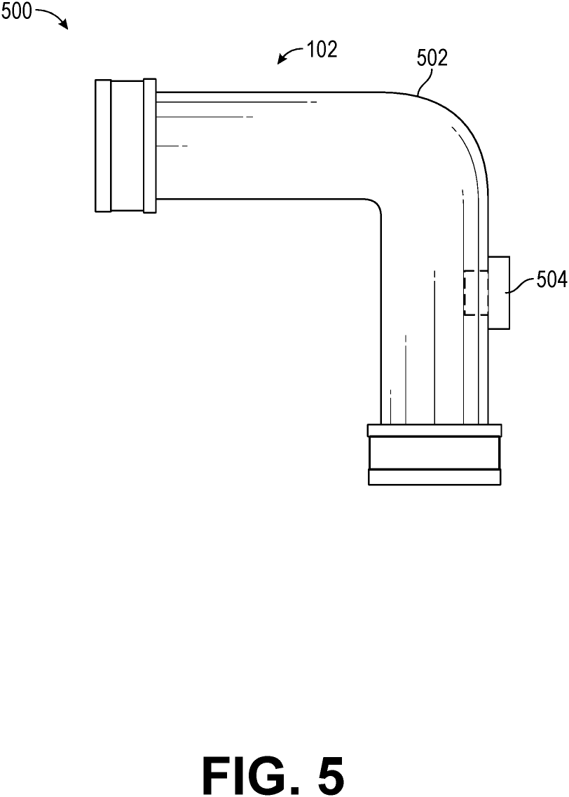

FIG. 5 depicts an additional example 500 of a fillable palm cooling device in accordance with one or more implementations. In this example, the fillable palm cooling device 102 includes a fillable tube 502 which is curved and a removable plug 504 which can be removed from the fillable tube 502 so that the fillable tube 502 can be filled with a cool substance, such as cool water.

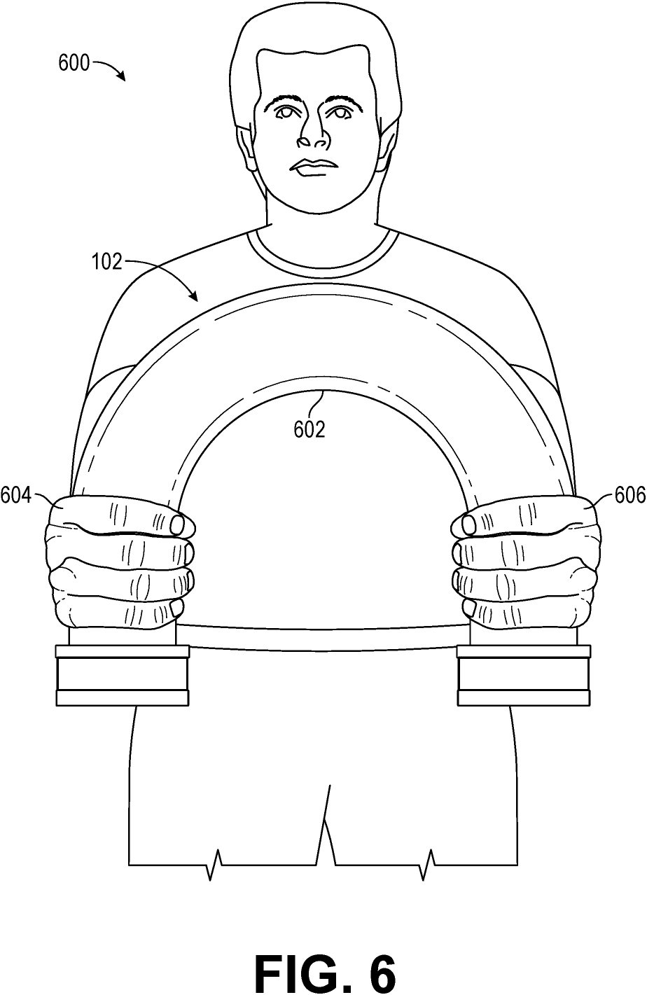

FIG. 6 depicts an additional example 600 of a fillable palm cooling device in accordance with one or more implementations. In this example, the fillable palm cooling device 102 is depicted with a fillable tube 602 which is curved, and is being held in both a first hand 604 and a second hand 606 of a person.

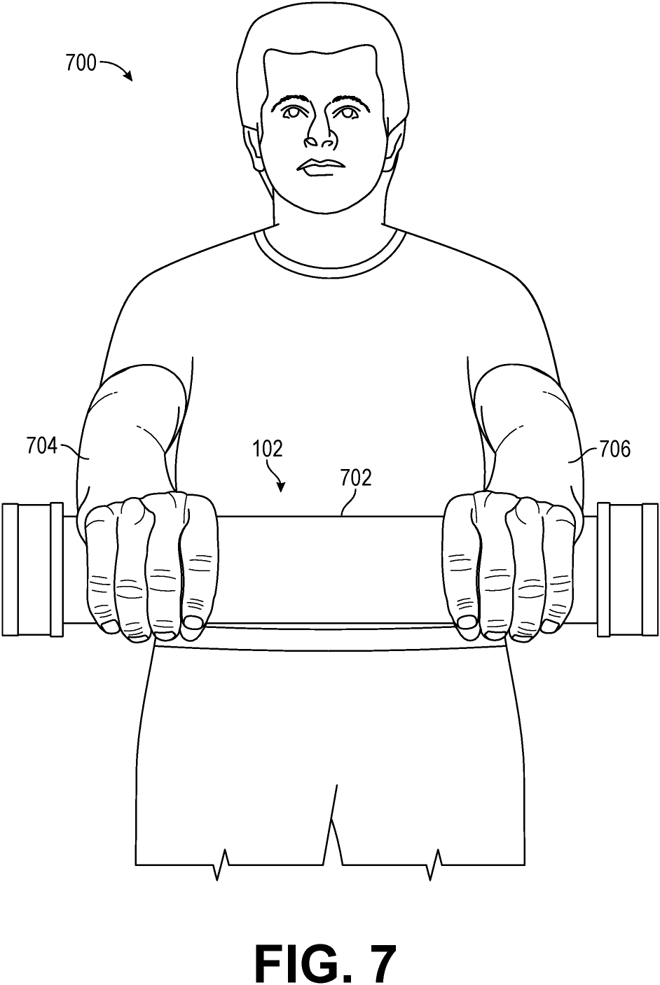

FIG. 7 depicts an additional example 700 of a fillable palm cooling device in accordance with one or more implementations. In this example, the fillable palm cooling device 102 is depicted with a fillable tube 702 which is straight, and is being held in both a first hand 704 and a second hand 706 of a person.

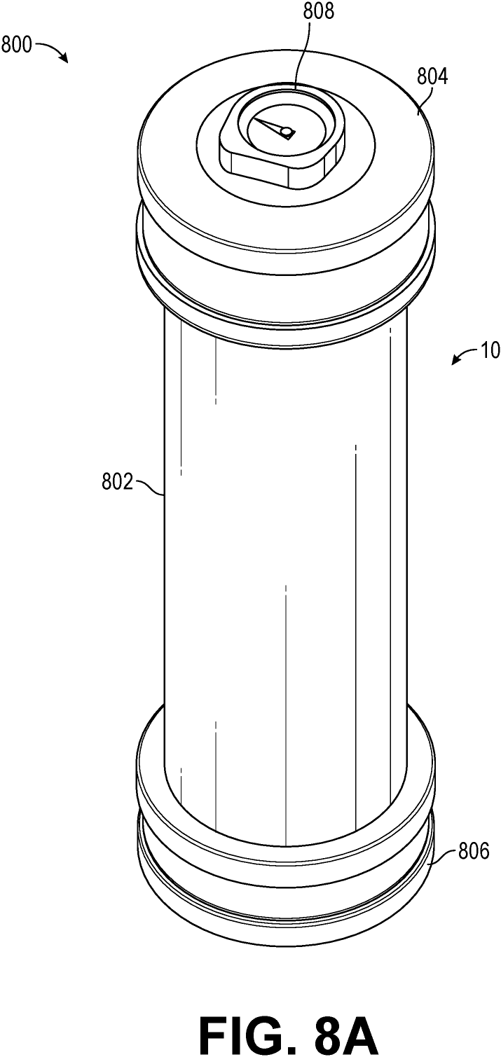

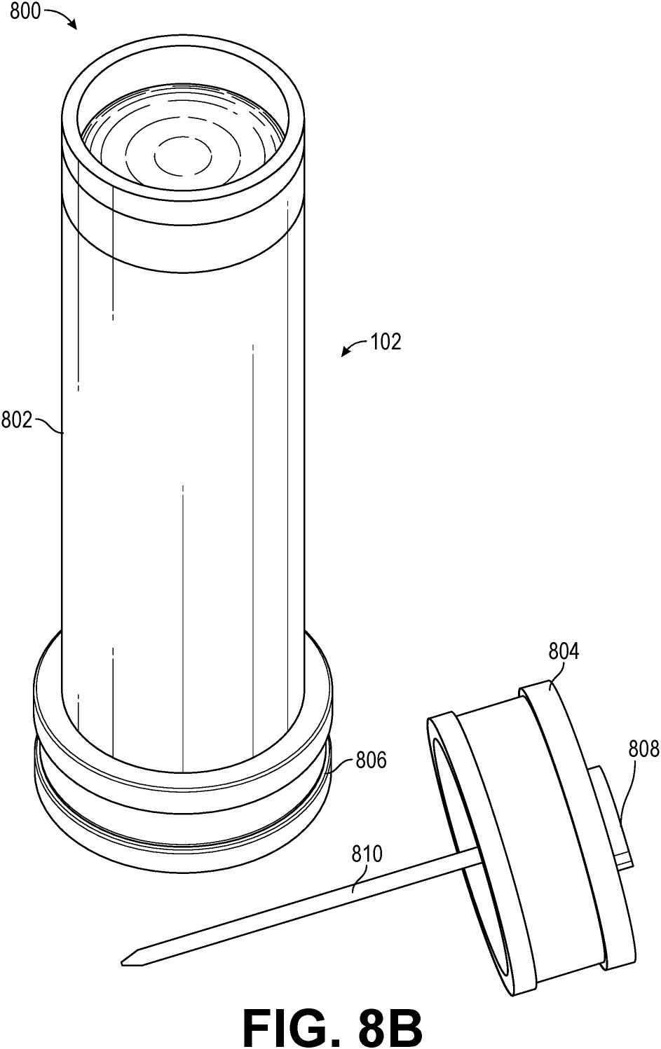

FIG. 8A depicts an additional example 800 of a fillable palm cooling device in accordance with one or more implementations.

In this example, the fillable palm cooling device 102 includes a fillable tube 802, a first endcap 804, and a second endcap 806. In one or more examples, one or both of the first endcap 804 and the second endcap 806 are removable so that the fillable tube 104 can be filled with a cool substance, such as cool water. Alternatively, in one or more implementations the fillable palm cooling device 102 includes a single removeable endcap (e.g., first endcap 804) that can be removed so that the fillable tube can be filled with a cool liquid.

In this example, the fillable palm cooling device 102 is further configured with an indicator mechanism 808. The indicator mechanism 808, in this example, is implemented as a thermometer and provides a notification of a current temperature of the fillable palm cooling device 102. Notably, the indicator mechanism 808 is positioned, in this example, on first endcap 804.

FIG. 8B depicts an additional example 800 of a fillable palm cooling device in accordance with one or more implementations. In this example, the fillable palm cooling device 102 of FIG. 8B is depicted with first endcap 804 removed from the fillable tube 802. As described throughout, the fillable tube 802 can be filled with a cool liquid when endcap 804 is removed. Endcap 804 is depicted with a temperature probe 810 of the indicator mechanism 808 that is used to measure the temperature of the cool liquid within the fillable tube 802.

While the fillable palm cooling device is described throughout as being filled with a cool substance in order to cool a user’s hands, the fillable palm cooling device can also be used to warm a user’s hands in one or more implementations. To do so, the fillable palm cooling device can be filled with a warm substance (e.g., warm water) and/or the fillable palm cooling device may be heated by an external heat source. Doing so warms the fillable tube. The warm tube of the device can then be held in both hands of a person in order to warm the person. For example, the person may hold the warmed device after cold exposure outdoors, during post operative recovery in the hospital, or if the person is hypothermic.

In one or more implementations, the fillable palm cooling device 102 further includes a cooling and/or heating mechanism that is configured to maintain the desired temperature of the device. The cooling and/or heating mechanism can be implemented in a variety of different ways. In one or more implementations, the fillable palm cooling device 102 includes a battery or electric powered cooling and/or heating mechanism which is configured to influence the temperature of the liquid inside the fillable palm cooling device, e.g., by reducing, maintaining, or increasing the temperature of the liquid. For example, a cooling mechanism may be configured to maintain the temperature of the liquid within the cooling temperature range. Alternately or additionally, the battery or electric powered cooling device can be configured to influence the temperature of the internal walls of the fillable tube which modifies the temperature of the liquid inside the device. In one or more implementations, one or both of the endcaps can be filled with fluid and frozen. The frozen fluid within the endcaps can enable the liquid within the device to stay cooler for a longer period of time.

What is claimed is:

1. A fillable palm cooling device comprising:
a fillable tube configured to be filled with a cool substance; and
at least a first endcap configured to be removed to enable the fillable tube to be filled with the cool substance, wherein the fillable tube is configured to be held in palms of both hands of a person to cool the palms of both hands of the person when the fillable tube is filled with the cool substance.

2. The fillable palm cooling device of claim 1, wherein the fillable tube is made from aluminum.

3. The fillable palm cooling device of claim 1, wherein the fillable tube is angled and includes a first portion configured to be held in a first palm of a first hand of the person and a second portion configured to be held in a second palm of a second hand of the person.

4. The fillable palm cooling device of claim 1, wherein the fillable palm cooling device further includes an indicator mechanism to indicate that a temperature of the fillable tube is within a cooling temperature range that is suitable for cooling the palms of both hands of the person.

5. The fillable palm cooling device of claim 1, wherein the fillable palm cooling device further includes an indicator mechanism to indicate that a temperature of the fillable tube is outside of a cooling temperature range that is suitable for cooling the palms of both hands of the person.

6. The fillable palm cooling device of claim 5, wherein the indicator mechanism is positioned on the fillable tube of the fillable palm cooling device.

7. The fillable palm cooling device of claim 5, wherein the indicator mechanism is positioned on the first endcap of the fillable palm cooling device.

8. The fillable palm cooling device of claim 1, wherein the fillable palm cooling device further comprises a second endcap configured to be removed to enable the fillable tube to be filled with the cool substance.

9. The fillable palm cooling device of claim 8, wherein the first endcap and the second endcap can be filled with a cool substance and frozen.

10. The fillable palm cooling device of claim 1, wherein the cool substance comprises cool water.

11. The fillable palm cooling device of claim 10, wherein the fillable palm cooling device can be re-cooled by emptying the cool water from the fillable palm cooling device, and re-filling the fillable palm cooling device with new cool water.

12. The fillable palm cooling device of claim 1, wherein the fillable palm cooling device can be re-cooled by placing the fillable palm cooling device in a refrigerator.

13. A fillable palm cooling device comprising:
a fillable tube configured to be filled with a substance, wherein the fillable tube is configured to be held in palms of both hands of a person to cool the palms of both hands of the person when the fillable tube is filled with the substance; and
a temperature indicator configured to indicate a temperature of the substance inside the fillable tube.

14. The fillable palm cooling device of claim 13, wherein the temperature indicator indicates whether the temperature of substance is within a cooling temperature range.

15. The fillable palm cooling device of claim 13, further comprising a cooling mechanism to maintain the temperature of the substance within a cooling temperature range.

16. The fillable palm cooling device of claim 13, further comprising a heating mechanism to maintain the temperature of the substance within a cooling temperature range.

17. The fillable palm cooling device of claim 13, wherein the temperature indicator is positioned on the fillable tube of the fillable palm cooling device.

18. The fillable palm cooling device of claim 13, wherein the temperature indicator is positioned on an endcap of the fillable palm cooling device.

19. The fillable palm cooling device of claim 13, wherein the substance comprises cool water.

20. The fillable palm cooling device of claim 13, wherein the substance comprises water, and wherein the fillable palm cooling device can be refrigerated to cool the water within the fillable tube.
